(19) United States
(12) Patent Application Publication
(10) Pub. No.: US 2003/0191374 A1
(43) Pub. Date: Oct. 9, 2003

(54) Computer-aided process for health screening management

(76) Inventor: Chwen-Keng Tsao, Taipei City (TW)

Correspondence Address: TROP PRUNER & HU, PC, 8554 KATY FREEWAY, SUITE 100, HOUSTON, TX, 77024, US

(21) Appl. No.: 10/118,769

(22) Filed: Apr. 9, 2002

(51) Int. Cl. A61B 5/00

(52) U.S. Cl. 600/300

(57) ABSTRACT

In a computer-aided process for health screening management employed in a health screening center with a plurality of examination rooms for conducting different examination items, basic data of the examinee is stored in a database of a computer system, and an identification code is assigned to the examinee. According to specified examination and facility parameters, the computer system selects one of the examination rooms that requires the least waiting time. The examinee is directed to the selected examination room. The identification code is inputted prior to entry of the examinee into the room for examination. This procedure is repeated until the examinee has completed all the examination items.

### BACKGROUND OF THE INVENTION

1. Field of the Invention

The invention relates to a process for health screening management, more particularly to a computer-aided process for health screening management that can effectively shorten the time required for carrying out a health screening.

2. Description of the Related Art

With the progress of society, people have higher living standards. However, while there are the amenities of civilization to enjoy, the fast tempo of modern life and high work pressure make people live under constant stress. This, combined with the pollution of air and water brought about by industrialization, and the lack of exercise, etc., has put people's health at stake. It can be seen that chronic diseases resulting from poor living habits occupy about four-fifths of the top ten list of killer diseases. Besides, there is a tendency for these diseases to occur in people of relatively young age. Research also shows that while people now have a longer life span than before, the rate of hospitalization is on the rise. In other words, the last years of many people are spent in hospitals. This implies that although people live longer, they do not take good care of their body and therefore do not have better health. Besides, the medical costs borne by the society where many people are unhealthy are immense.

Therefore, to conduct regular health screening so as to know the current health condition of our body and to discover any latent diseases at an earlier date is necessary so that preventive medical services or treatment can be timely sought to prevent breakout or worsening of the diseases.

However, as the basic health screening services now offered by hospitals or health clinics often require examinees to stay overnight for examination, it is quite difficult for busy career people to find the spare time. It will be desirable and convenient to most people if the time required to conduct health screenings can be shortened. A basic health screening generally includes a number of items, such as blood tests, X-ray tests, sonography, etc. Each of these items may be further divided into a number of sub-items. For instance, blood tests may include routine blood examination, blood sugar tests, liver function tests, blood lipids tests, etc. As the facilities of a hospital or clinic are limited and are located in different parts of the building for use by both in-patients and out-patients, if all of the items, including the sub-items, are conducted one by one in sequence, the examinee may have to wait for an unnecessary time interval between tests/examinations. This means that most of the examinee's time at the hospital or clinic is spent waiting to be examined. If the waiting time can be shortened, the time required to carry out the health screening will be undoubtedly reduced.

### SUMMARY OF THE INVENTION

Therefore, the main object of the present invention is to provide a computer-aided process for health screening management that can effectively shorten the time required for carrying out a health screening.

Accordingly, a computer-aided process for health screening management of this invention is adapted to be employed in a health screening center that includes an examination area with a plurality of examination rooms where different health screening items can be performed on an examinee, and a computer system linked to the examination rooms. The computer-aided process includes:

(a) storing basic data of the examinee in a database of the computer system;

(b) assigning an identification code to the examinee;

(c) inputting the identification code prior to entry of the examinee into any one of the examination rooms;

(d) with the use of the computer system and in accordance with specified examination and facility parameters, selecting one of the examination rooms that is responsible for an examination item yet to be performed on the examinee and that requires the least amount of waiting time, and directing the examinee to the selected one of the examination rooms;

(e) upon completing examination in the selected one of the examination rooms, inputting the identification code, and determining whether all of the examination items have been performed on the examinee with the use of the computer system; and

(f) repeating steps (d) and (e) until it has been determined that all of the examination items have been performed on the examinee.

### BRIEF DESCRIPTION OF THE DRAWINGS

Other features and advantages of the present invention will become apparent in the following detailed description of the preferred embodiment with reference to the accompanying drawings, of which:

FIG. 1 illustrates a layout of a health screening center that employs the preferred embodiment of a computer-aided process for health screening management according to the present invention;

FIG. 2 is a list of stations and rooms of the health screening center, and examination items provided by the same;

FIG. 3 illustrates a layout of a before-meal examination area of the health screening center;

FIG. 4 illustrates a layout of an after-meal examination area of the health screening center;

FIG. 5 is a flowchart of the computer-aided process of the preferred embodiment; and

FIG. 6 is a table that lists the examination times and the number of different stations and rooms in an exemplary model that employs the computer-aided process of the preferred embodiment.

### DETAILED DESCRIPTION OF THE PREFERRED EMBODIMENT

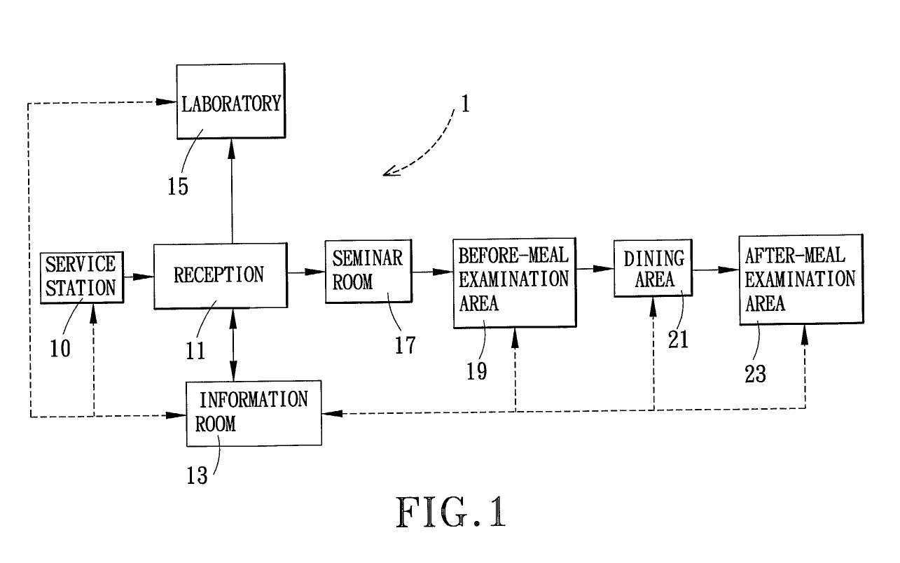

FIG. 1 shows a layout of a health screening center 1 employing the preferred embodiment of a computer-aided process for health screening management according to the present invention. The health screening center 1 includes a service station 10, a reception 11, a seminar room 17, an information room 13, a laboratory 15, a before-meal examination area 19, a dining area 21, and an after-meal examination area 23.

The service station 10 is provided for confirming the identities of examinees, making appointments for health screenings, receiving payments, and for advising the examinees of the things to be kept in mind before and during the health screening. In addition, if it is the first time for the examinee to undergo examination in the health screening center 1, the examinee is requested to fill up a basic data form for creating a basic data file. In this embodiment, in order to shorten the length of time to conduct the health screening, certain procedures pertaining to the health screening are carried out beforehand. For instance, the examinee is given a stool collector, a health evaluation questionnaire, etc., for completion before the actual health screening. In other words, after the examinee has confirmed the appointment and has attended to payment of the relevant fees, the staff at the service station 10 will give the examinee a health evaluation questionnaire for completion at home and a stool collector for collecting stool sample, which are to be submitted on the day of the actual health screening. The health evaluation questionnaire includes questions relating to personal life style, working conditions, eating habits, personal medical history, family medical history, current physical condition, etc., and is intended to shorten the interview with the doctor. In addition, the staff at the service station 10 will advise the examinee of the things to be kept in mind before and during the health screening, for instance, the examinee should fast eight hours before the actual health screening, so that the examinee can be well prepared for the same.

The examinee first goes to the reception 11 at the appointed time on the day of the health screening. At the reception 11, the identity of the examinee is confirmed, and the examinee is given a magnetic card containing an exclusive identification code. The aforesaid health evaluation questionnaire and the stool collector are also collected. Then, the examinee is directed to the seminar room 17 where the health screening process will be explained to the examinee. The collected health evaluation questionnaire is encoded, and the data are printed out to yield an electronic medical history for reference by the doctor. On the other hand, the stool collector, which contains the stool sample, is passed on to the laboratory 15 for analysis.

For purposes of computer-aided management, a computer system is disposed in the information room 13, and a computer network is established for on-line connection among the information room 13, the service station 10, the reception 11, the laboratory 15, the before-meal examination area 19, the dining area 21, and the after-meal examination area 23 so that data can be transmitted and collected thereamong. The basic data filled in by the examinee at the service station 10 is sent to the information room 13 for storage so that the basic data can be quickly retrieved from the information room 13 during the course of the health screening, thereby saving time. Moreover, in order to facilitate electronic data management, the health evaluation questionnaire is designed to be readable and processable directly by an optical card scanner so that mechanical inputting of data can replace manual inputting of data to thereby reduce the data inputting time.

At the seminar room 17, the health screening process and the purposes of the examination items to be conducted are explained to the examinee so as to provide the examinee with a better understanding of the whole health screening process. Thereafter, the examinee is directed to the before-meal examination area 19 where the health screening process will begin.

Conventional health screenings require examinees to fast for a relatively long time period prior to the examinations, which may not suit some people. In the present invention, the examination items are divided into those that require fasting and those that do not. Thus, the examinee can be offered a meal immediately after completion of the examination items that require fasting so as to dispel any hunger pangs that the examinee may suffer. Therefore, in this embodiment, those examination items that require the examinee to fast are prioritized over those that do not, and are conducted in the before-meal examination area 19, while the rest are conducted in the after-meal examination area 23.

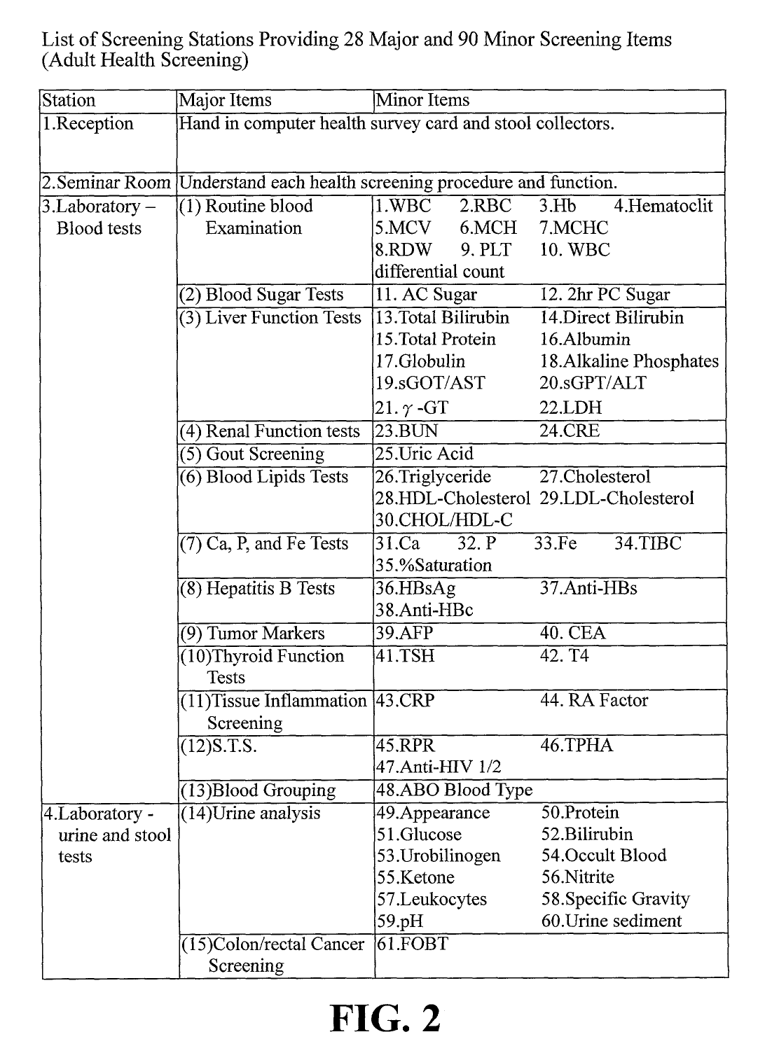

With further reference to FIG. 2, the basic examination items that are adopted for classification in this embodiment include ninety minor items which can be grouped into twenty-eight major items that include: (1) routine blood tests; (2) blood sugar tests; (3) liver function tests; (4) renal function tests; (5) gout screening; (6) blood lipids tests; (7) Ca, P, and Fe tests; (8) hepatitis B tests; (9) tumor markers; (10) thyroid function tests; (11) tissue inflammation screening; (12) sexually transmitted disease screening (S.T.S.) ; (13) blood grouping; (14) urine analysis; (15) colon/rectal cancer screening; (16) general examination; (17) ears, nose and throat (E.N.T.) examination; (18) abdominal sonography examination; (19) chest X-ray test; (20) abdomen X-ray test; (21) upper gastrointestinal X-ray test; (22) audio acuity test; (23) pulmonary function examination; (24) ophthalmic and ocular tension tests; (25) bone mineral density screening; (26) electrocardiogram examination; (27) cervical carcinoma test; and (28) doctor conclusions/physical examination. Of the aforesaid major items, items (1) to (13) relate to blood tests, while items (14) to (15) relate to urine and stool tests.

The laboratory 15 is the place where blood tests, stool tests, and urine analysis are conducted. After the examinee has registered at the reception 11, the examinee is directed to the laboratory 15 where blood and urine samples are obtained. Since the laboratory 15 is linked to the information room 13, once the results of the tests and analyses are generated, the same can be transmitted to the information room 13 for integration with the examination data of the examinee.

Of the twenty-eight major items, items (1) to (15) are completed at the laboratory 15, and only items (16) to (21) require fasting prior to examination. The before-meal examination area 19 in this embodiment is designed to cover these items (16) to (21).

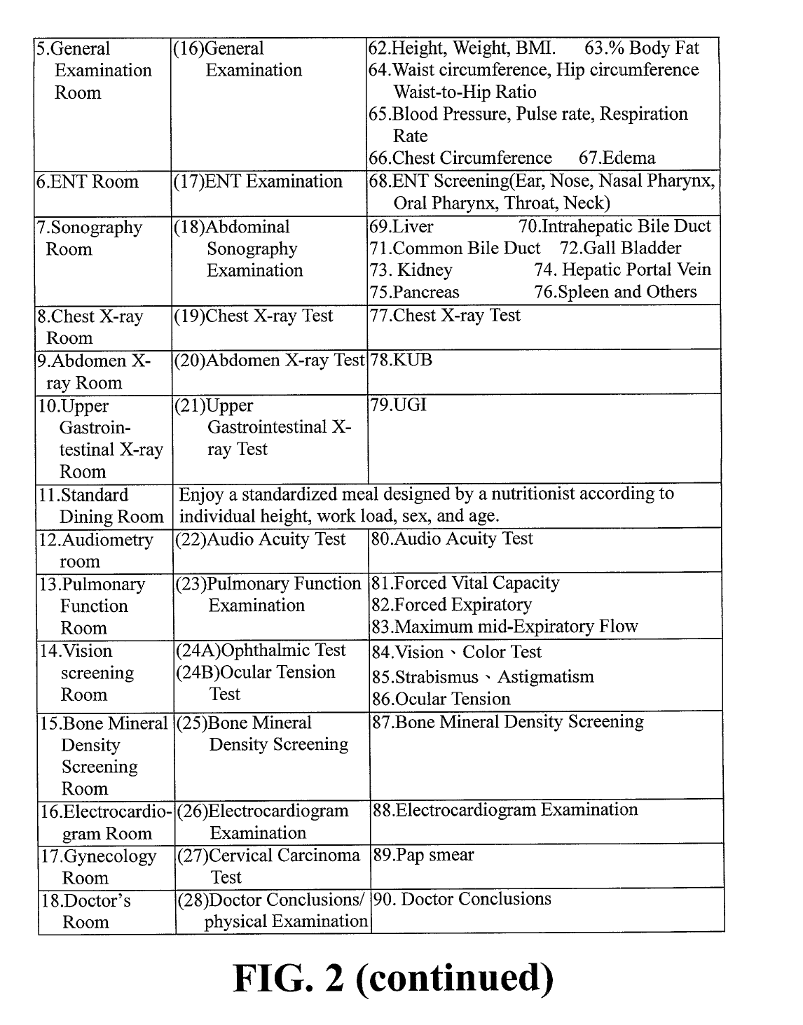

FIG. 3 illustrates the layout of the before-meal examination area 19 according to this embodiment. The before-meal examination area 19 is shown to be divided into a general examination room 31, an ENT room 32, a chest X-ray room 33, an abdomen X-ray room 34, a sonography room 35, and an upper gastrointestinal X-ray room 36. The items of examination to be conducted at the general examination room 31 include height, weight, body mass index, body fat percentage, blood pressure, pulse rate, respiration rate, chest circumference, waist circumference, hip circumference, waist-to-hip ratio, and edema. Since the items of examination to be conducted at the general examination room 31 are many in number, for the purpose of effective management, the general examination items may be divided into two groups, in which examinations pertaining to height, weight, body mass index, and body fat percentage are conducted at a first general examination room 311, whereas the rest of the examinations are conducted at a second general examination room 312. The ENT room 32 is responsible for the examination of the ears, nose, nasal pharynx, oral pharynx, throat, and neck. The chest X-ray room 33, the abdomen X-ray room 34, and the upper gastrointestinal X-ray room 36 are responsible for X-ray examinations of the chest, the abdomen and the upper gastrointestinal system, respectively. The sonography room 35 is responsible for sonography examination of the liver, the intrahepatic bile duct, the common bile duct, the gall bladder, the kidneys, the hepatic portal vein, the pancreas, the spleen, and other organs. Each of the examination rooms 311, 312, 32, 33, 34, 35, 36 is equipped with an identification device, such as a card reader, and an LCD display panel linked to the information room 13 to facilitate optimized examination scheduling, which will be described in detail hereinafter.

After completing the examinations in the before-meal examination area 19, the examinee is directed to the dining area 21 where the examinee can enjoy a standardized meal designed by a nutritionist according to the height, work load, sex, age, etc., of the examinee. Thereafter, the examinee can proceed to the after-meal examination area 23 to conduct examination of items (22) to (28) of the aforesaid major items.

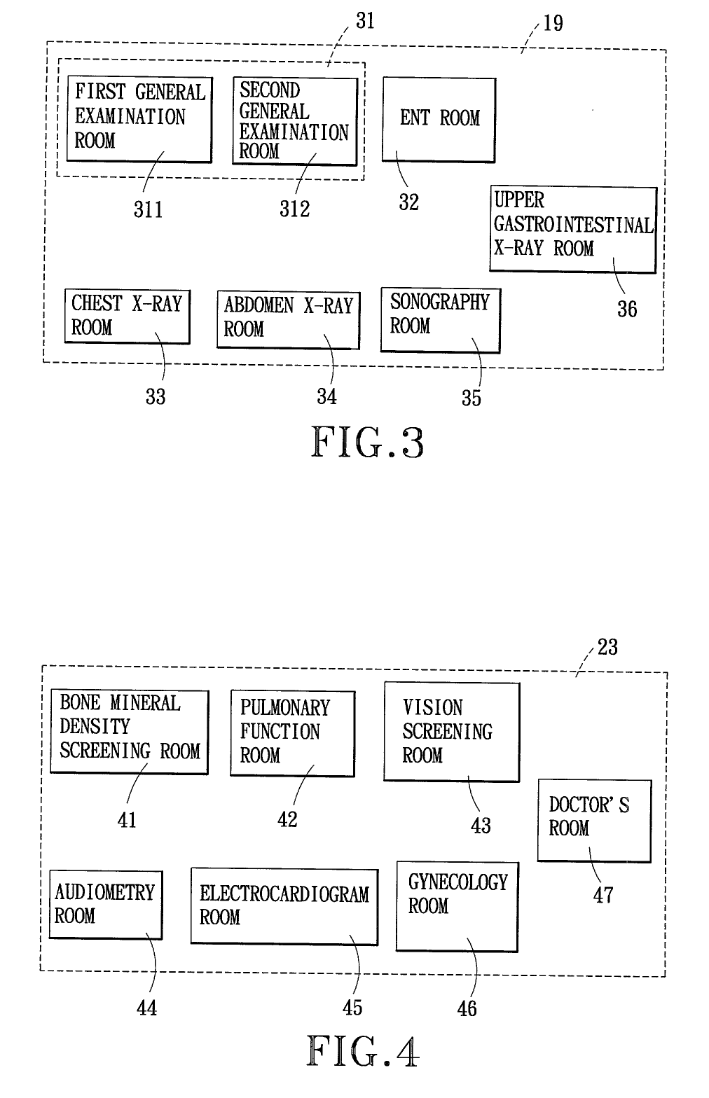

FIG. 4 illustrates the layout of the after-meal examination area 23 according to this embodiment. The after-meal examination area 23 is shown to be divided into a bone mineral density screening room 41, a pulmonary function room 42, a vision screening room 43, an audiometry room 44, an electrocardiogram room 45, a gynecology room 46, and a doctor's room 47. At the doctor's room 47, the doctor makes a conclusion and provides suggestions based upon the health screening results integrated via the information room 13, and explains to the examinee the screening results. Since the doctor can review the electronic medical history of the examinee, the interview time can be shortened to effectively reduce duration of the health screening process. Like the before-meal examination area 19, the instruments in the rooms 41 to 47 of the after-meal examination area 23 are linked to the information room 13 so that examination results can be sent to the information room 13 for integration. Moreover, the entrance of the after-meal examination area 23 and those of the examination rooms 41 to 47 are respectively provided with identification devices and LCD display panels linked to the information room 13. Therefore, with the exception of the doctor's room 47, examination in the rooms 41 to 46 can be scheduled so as to shorten the waiting time effectively.

The flow of the preferred embodiment of the computer-aided process for health screening management according to the invention will now be described in greater detail in the succeeding paragraphs.

In this embodiment, cross-scheduling is adopted to arrange the sequence of the examinations the examinee is supposed to have. In other words, the examinations are not conducted according to the sequence of the examination items set forth in the list shown in FIG. 2. The examination items that require the least amount of waiting time are prioritized. Each of the examination rooms 31 to 36 is linked to the information room 13, which controls the flow of the health screening process for each of the examinees. The information room 13 arranges the order of the examinations according to the waiting times estimated thereby. Once the examinee has completed examination at one of the examination rooms, the information room 13 will immediately calculate and find out the examination item that requires the least amount of waiting time among all of the examination items that are yet to be completed, and will instruct the examinee to move to the appropriate examination room. As such, the examinee need not waste too much time waiting between examinations to be conducted at rooms 31 to 36, and optimum utilization of resources can be achieved. The information room 13 determines the estimated waiting times at rooms 31 to 36 according to the number of examinees waiting at the respective examination room and the time required to conduct the respective examination. To illustrate, it is assumed that the examinee has completed the general examination, the ENT examination, and the sonography examination, with the upper gastrointestinal X-ray test not being considered in the meantime (to be explained hereinafter). It is further assumed that the examination at the chest X-ray room 33 takes about 1 minute and there are five other examinees waiting, whereas the examination at the abdomen X-ray room 34 takes about 2 minutes and there are three other examinees waiting. In this case, the examinee will be instructed to wait at the chest X-ray room 33 for examination. In order to enable the information room 13 to monitor and control movement of the examinee in real time, an identification device (such as a card reader) which is linked to the information room 13 and which can read the identification code on the examinee's magnetic card, and an LCD display panel which can display the designated examination room in real time are installed at the entrance of the before-meal examination area 19 and at the entrance of each of the examination rooms 31 to 36. As such, when the examinee logs in at the entrance of the before-meal examination area 19 using the magnetic card, the information room 13 selects the examination room that requires the least amount of waiting time, and displays the same on the computer screen and the corresponding LCD display panel, thereby instructing the examinee to go to the selected examination room. On entering the designated examination room, the examinee needs to log in by swiping the magnetic card at the identification device so that the information room 13 can confirm that the examinee has entered the selected examination room. After completion of the examination item(s) at the selected examination room, since the examination instruments in each of the examination rooms are linked to the information room 13, the results of the examination can be quickly transmitted to the information room 13 for integration. Together with the examination results, the identification code of the examinee is also sent to the information room 13 so that the latter is able to readily confirm the identity of the examinee and to integrate the examination results correctly. When the examinee leaves the selected examination room, the examinee is once again requested to input the identification code through the use of the magnetic card to thereby log out. Hence, the information room 13 can be certain that the examinee has completed examination at the selected examination room, and can concurrently display on both the computer screen and the LCD display panel the next examination room that takes the least amount of waiting time to thereby instruct the examinee to proceed to and wait at the next designated examination room, where the aforesaid log-in and log-out operations are repeated. When the examinee logs out, the information room 13 will select, from among the examination rooms in which the examinee has yet to be examined, one that requires the least waiting time and will instruct the examinee to proceed to the next selected examination room for examination. The process is repeated until the examinee has completed examinations at all of the examination rooms. By using the computer system at the information room 13 to dynamically adjust the number of examinees waiting at each of the examination rooms, all the examinees do not have to waste too much time waiting, thereby effectively shortening the time to do health screenings.

It is noted that some examination items may affect the results of the subsequent examinations. For instance, the intake of a developing agent necessary for the upper gastrointestinal X-ray test will affect the results of chest and abdomen X-ray tests. Hence, in this embodiment, a parameter of cross-scheduling by the information room 13 is set to have the upper gastrointestinal X-ray test arranged as the last examination item in the before-meal examination area 19.

In addition, since the examination times at the examination rooms 31 to 36 differ, the number of the examination rooms 31 to 36 can vary depending upon actual requirements so that the waiting time can be more effectively shortened.

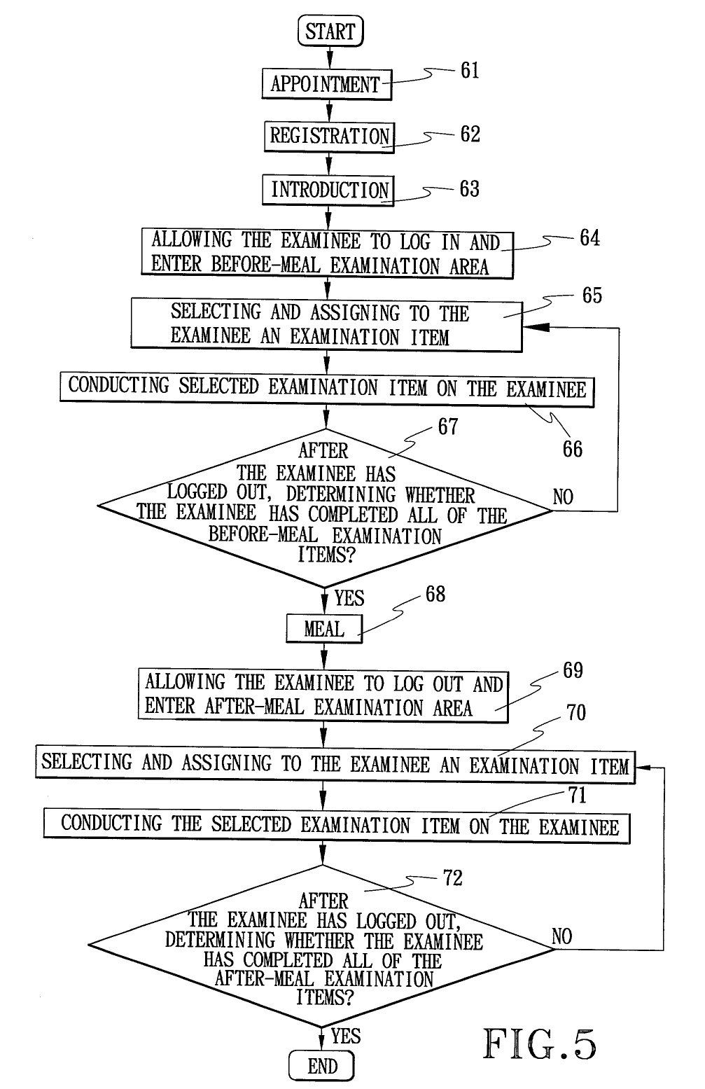

Based upon the aforesaid and with further reference to FIG. 5, the flow of the entire computer-aided process according to the present invention will now be described in the following paragraphs.

In step 61, the examinee goes to the service station 10 to make an appointment for the health screening, confirms the appointment time, and obtains the health evaluation questionnaire, a stool collector, and other items that the examinee can complete on his/her own. In this embodiment, the examinee has to personally go to the service station 10 to attend to the above. The examinee can also make the appointment by telephone, via the Internet, or by any other means.

In step 62, the examinee reports at the reception 11 on the date of the appointment, makes the necessary arrangements, such as submitting the completed health evaluation questionnaire and the stool collector with the stool sample, and obtains a magnetic card containing an exclusive identification code, etc. A blood sample of the examinee will be obtained at the laboratory 15 for analysis.

In step 63, staff at the health screening center 1 will explain to the examinee the procedures of the examinations and the things to be kept in mind. The examinee is also asked for a urine sample for analysis.

In step 64, the examinee enters the before-meal examination area 19 after logging in using the magnetic card.

Upon logging in, the flow proceeds to step 65, where the information room 13, according to specific parameters (i.e., the parameter that the upper gastrointestinal X-ray test is specified to be the last before-meal examination item, and the parameter that the examination item that takes the least amount of waiting time is prioritized), designates an examination room for the examinee. Hence, all the examinees at the before-meal examination area 19 can be allocated to different examination rooms to thereby shorten the waiting time.

In step 66, the examinee logs in at the designated examination room for examination. Upon completion, the examination results will be sent to the information room 13 for integration. In step 67, the examinee uses the magnetic card to log out, whereupon the information room 13 will determine whether the examinee has completed all of the before-meal examination items.

In step 67, if it is determined that the examinee has yet to complete all of the before-meal examination items, step 65 is repeated, and the information room 13 selects one among the examination rooms in which the examinee has yet to be examined, and instructs the examinee to go there for examination. In this way, the examinee can complete all of the examination items smoothly.

If it is determined in step 67 that the examinee has completed all of the before-meal examination items, the flow proceeds to step 68, where the examinee is directed to the dining area 21 to have a standardized meal, which is designed by a nutritionist based upon the sex, age, ideal weight, and the work load of the examinee as noted from the completed health evaluation questionnaire to serve as a meal model for the examinee's reference.

After the meal, the flow goes to step 69, where the examinee proceeds to the after-meal examination area 23 to log in for conducting the subsequent examination items in a manner similar to that described hereinabove in connection with steps 64 to 67.

Upon the logging in of the examinee, the information room 13 selects an examination room according to specific parameters in step 70, and instructs the examinee to go there.

Thereafter, in step 71, the examinee logs in at the designated examination room for examination. After completion of the examination, the flow proceeds to step 72, where the examinee logs out, and the information room 13 determines whether the examinee has completed all of the after-meal examination items. In the negative, step 70 is repeated to continue with the examination process. Otherwise, it is determined that examination is completed. Since all the data are computerized, the examination results can be printed out for the examinee upon completion of all of the examination items.

In order to effectively control the flow of examinees and to effectively utilize human and facility resources, in this embodiment, health screenings are available both in the morning and in the afternoon. Examinees who have made appointments with the health screening center 1 should report to the reception 11 within the appointed time. For instance, if the examinee makes an appointment for a morning health screening session, the examinee is required to arrive between 8:00 a.m. and 8:30 a.m. so as to control the flow of examinees for the morning.

In order to prove that the computer-aided process according to this invention can indeed shorten the waiting time during health screening and can serve a larger number of examinees at the same time, the process of this invention is exemplified below using a model in which ninety examinees undergo health screenings within a half-day session.

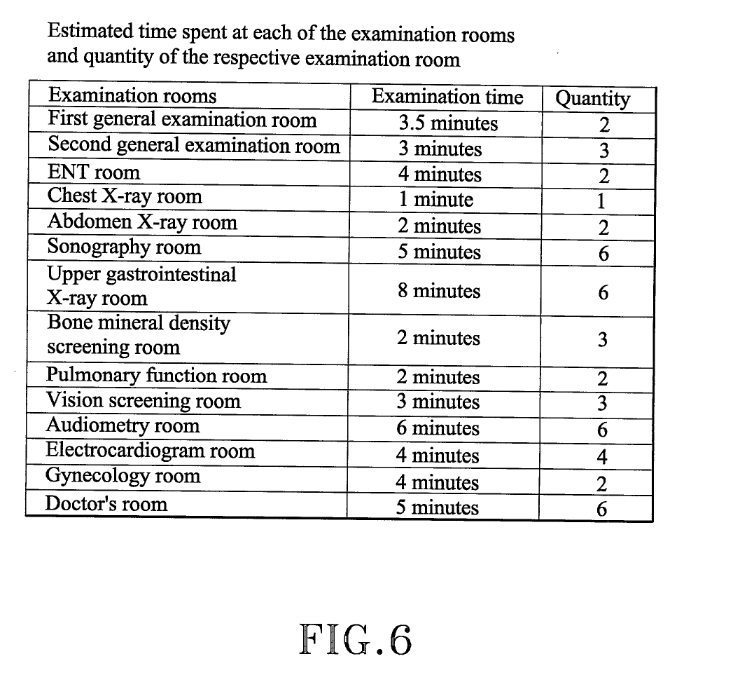

The process flow begins after blood samples are obtained. Referring to FIG. 6, experiments show that the general examination at the first general examination room 311 takes about 3.5 minutes, and that the general examination at the second general examination room 312 takes about 3 minutes. ENT examination at the ENT room 32 takes about 4 minutes. The chest X-ray test at the chest X-ray room 33 takes about 1 minute. The abdomen X-ray test at the abdomen X-ray room 34 takes about 2 minutes. The sonography examination at the sonography room 35 takes about 5 minutes. The upper gastrointestinal X-ray test at the upper gastrointestinal X-ray room 36 takes about 8 minutes. The bone mineral density screening at the bone mineral density screening room 41 takes about 2 minutes. The pulmonary function examination at the pulmonary function room 42 takes about 2 minutes. The ophthalmic and ocular tension tests at the vision screening room 43 take about 3 minutes. The audio acuity test at the audiometry room 44 takes about 6 minutes. The electrocardiogram examination at the electrocardiogram room 45 takes about 4 minutes. The Pap smear at the gynecology room 46 takes about 4 minutes. The doctor's conclusion of the health screening at the doctor's room 47 takes about 5 minutes. On the whole, under normal circumstances and without any undue delay, the entire health screening procedure takes about 52.5 minutes (not including the time to obtain the blood sample) to complete. Therefore, if the waiting time can be considerably shortened, the overall health screening time can be reduced effectively. In addition, since the times required for conducting the tests/examinations at the different examination rooms vary, more rooms are provided for those tests/examinations that take a comparatively longer amount of time to complete. In this model, there is one chest X-ray room 33, two first general examination rooms 311 assigned to conduct the first part of the general examinations, two ENT rooms 32, two abdomen X-ray rooms 34, two pulmonary function rooms 42, two gynecology rooms 46, three second general examination rooms 312 assigned to conduct the second part of the general examinations, three bone mineral density screening rooms 41, three vision screening rooms 43, four electrocardiogram rooms 45, six sonography rooms 35, six upper gastrointestinal X-ray rooms 36, six audiometry rooms 44, and six doctor's rooms 47. In addition, in order to facilitate altogether 61 tests/analyses of blood, urine and stool samples of ninety examinees, the laboratory 15 is equipped with one automatic hematology analyzer, two automatic clinical chemistry analyzers, eleven automatic immunoassay analyzers, one immunological FOBT analyzer, and one urine analyzer. It is noted that the aforesaid quantity of laboratory instruments is planned for optimum efficiency and such that test/analysis results can be obtained within four hours. Under the aforesaid conditions, it has been proven that ninety examinees can have their health screenings completed within four hours.

Preferably, a single identification device is provided at a convenient location. In addition, the logging in and logging out operations can be assisted by the staff at the health screening center 1.

In view of the aforesaid, the present invention, which employs computerized management to cross-schedule examinations and to integrate the examination results, can shorten waiting time to a considerable extent and can quickly integrate data, thereby effectively reducing the duration of the health screening. On the other hand, computerized management can serve more examinees concurrently to thereby reduce costs. The popularity of health screenings can also be enhanced to help people better understand their health conditions and to enable people with latent ailments to take preventive measures, which in turn can help save medical expenses.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

I claim:

1. A computer-aided process for health screening management adapted to be employed in a health screening center that includes an examination area with a plurality of examination rooms where different health screening items can be performed on an examinee, and a computer system linked to the examination rooms, said computer-aided process comprising:
(a) storing basic data of the examinee in a database of the computer system;
(b) assigning an identification code to the examinee;
(c) inputting the identification code prior to entry of the examinee into any one of the examination rooms;
(d) with the use of the computer system and in accordance with specified examination and facility parameters, selecting one of the examination rooms that is responsible for an examination item yet to be performed on the examinee and that requires the least amount of waiting time, and directing the examinee to the selected one of the examination rooms;
(e) upon completing examination in the selected one of the examination rooms, inputting the identification code, and determining whether all of the examination items have been performed on the examinee with the use of the computer system; and
(f) repeating steps (d) and (e) until it has been determined that all of the examination items have been performed on the examinee.

2. The computer-aided process of claim 1, wherein, in step (e), results of the examination in the selected one of the examination rooms are sent to the computer system together with the identification code of the examinee for data integration by the computer system.

3. The computer-aided process of claim 1, the examination items including before-meal examination items, which require fasting prior to examination, and after-meal examination items, which do not require fasting, wherein the examination parameters include one in which the before-meal examination items should be performed prior to the after-meal examination items.

4. The computer-aided process of claim 3, further comprising the step of allowing the examinee to take a meal after completion of the before-meal examination items.

5. The computer-aided process of claim 3, wherein the examination parameters further include one in which the examination items that will interfere with other ones of the examination items are given least priority.

6. The computer-aided process of claim 5, wherein the examination items which are given the least priority include those that require intake of an agent.

7. The computer-aided process of claim 6, wherein the before-meal examination items include: general examination; ears, nose and throat examination; chest X-ray test; abdomen X-ray test; sonography examination; and upper gastrointestinal X-ray test.

8. The computer-aided process of claim 7, wherein the upper gastrointestinal X-ray test requires the intake of a developing agent.

9. The computer-aided process of claim 5, wherein the after-meal examination items include: cervical carcinoma test; pulmonary function examination; electrocardiogram examination; audio acuity test; ophthalmic and ocular tension tests; and bone mineral density screening.

10. The computer-aided process of claim 1, further comprising, prior to step (c), the step of requiring the examinee to provide at least one of stool, blood and urine samples for analysis.

11. The computer-aided process of claim 1, wherein, in step (d), the waiting time is determined by the computer system according to the time required to conduct the examination item and the number of examinees waiting to be examined.
